(19) United States
(12) Patent Application Publication
(10) Pub. No.: US 2007/0129328 A1
(43) Pub. Date: Jun. 7, 2007

(54) Pharmaceutical compositions of neurokinin receptor antagonists and cyclodextrin and methods for improved injection site toleration

(76) Inventors: Wayne Boettner, Noank, CT (US); Christine Miskell, Colchester, CT (US)

Correspondence Address: PHARMACIA & UPJOHN, 7000 Portage Road, KZO-300-104, KALAMAZOO, MI, 49001, US

(21) Appl. No.: 10/587,808

(22) PCT Filed: Jan. 6, 2005
(86) PCT No.: PCT/IB05/00020

§ 371 Date: Jul. 28, 2006

Related U.S. Application Data
(60) Provisional application No. 60/540,644, filed on Jan. 30, 2004.

(51) Int. Cl. A61K 31/724; A61K 31/4745

(52) U.S. Cl. 514/58; 514/305

(57) ABSTRACT

This invention relates to pharmaceutical compositions for improving anesthesia recovery and preventing nausea and emesis and a method for improved injection site tolerance. In particular, the invention is directed to pharmaceutical compositions with an improved injection site toleration comprising an effective amount of a neurokinin receptor antagonist with a pharmaceutically acceptable cyclodextrin. The invention is also directed to pharmaceutical compositions of the compound of Formula (I), wherein R² is selected from the group consisting of methyl, ethyl, isopropyl, sec-butyl and tert-butyl. The invention is also directed to pharmaceutical compositions of the compound of Formula Ia, and cyclodextrins and methods for improved injection site toleration thereof.

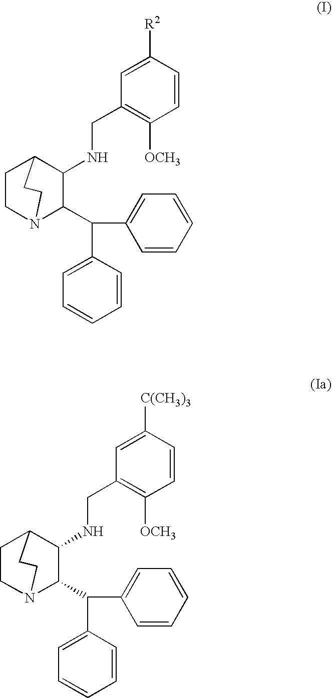

### FIELD OF INVENTION

The present invention is directed to pharmaceutical compositions containing cyclodextrins for improved injection site toleration and neurokinin receptor (NK-1) antagonists. The invention is also directed to pharmaceutical compositions of the compounds of Formula I, wherein R² is selected from the group consisting of methyl, ethyl, isopropyl, sec-butyl and tert-butyl.

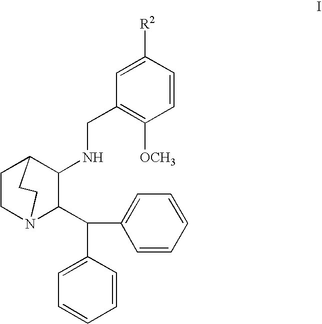

In particular, the invention is directed to pharmaceutical compositions of the compound of Formula Ia, (2S,3S)-2-benzhydryl-N-(5-tert-butyl-2-methoxybenzyl)quinuclidin-3-amine, and cyclodextrins for improved injection site toleration.

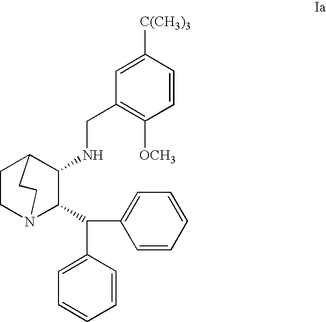

### BACKGROUND OF THE INVENTION

The compounds of Formula I or Ia are the subject of U.S. Pat. No. 5,807,867, U.S. U.S. Pat. No. 6,222,038 and U.S. Pat. No. 6,255,320. The preparation of compounds of Formula I and Ia are described therein. The compound of Ia may also be prepared as described in the co-pending U.S. provisional application No. 60/541,323, commonly owned and assigned to Pfizer, Inc. U.S. Pat. No. 5,393,762 also describes pharmaceutical compositions and treatment of emesis using NK-1 receptor antagonists. Co-pending U.S. provisional application No. 60/540,697, commonly owned and assigned to Pfizer, Inc., described a method of improving anesthesia recovery in patients by administering the compound of Formula Ia or Ia. The text of the aforementioned applications, patents and all other references cited in this specification are hereby incorporated by reference in their entirety.

Prevention and/or treatment of emesis has focused on substances that block or hinder neurokinin receptor (NK-1) activity. These substances are known as neurokinin receptor antagonists. There are numerous neurokinin receptor antagonists known in the art. Neurokinin antagonists include, but are not limited to, piperizino derivatives (U.S. Pat. No. 5,798,359), trypthophan urea (U.S. Pat. No. 5,869,489), spiro-substituted azacycles (U.S. Pat. No. 5,869,496), various amino acid derivatives (U.S. Pat. No. 5,849,918), arylglycinamide derivatives (U.S. Pat. No. 6,124,296), therapeutic heterocycles (U.S. Pat. No. 6,124,279), aromatic amine compounds (U.S. Pat. No. 5,686,609), quaternary imodium salts (U.S. Pat. No. 5,674,881), and other neurokinin receptor antagonists known to those of skill in the art.

Administering NK-1 antagonists, however, present various problems with regard to injection site tolerance (e.g., irritability of subject, irritation, inflammation, swelling, and/or redness of the site). Although there have been numerous studies with regard to improving injection site tolerance through the use of various substances, none of these studies, however, have focused on neurokinin receptor antagonist administration.

It was determined that improved injection site toleration was realized by the addition of a cyclodextrin to the pharmaceutical composition containing a neurokinin receptor antagonist. Cyclodextrins are cyclic oligosaccharides. There are three main cyclodextrins: α-cyclodextrin is composed of a ring of six glucose residues; β-cyclodextrin is composed of a ring of seven glucose residues; and γ-cyclodextrin is composed of a ring of eight glucose residues. Typically, cyclodextrins are formed by the action of an amylase on starch. Cyclodextrins typically vary in shape and size, but are, generally, defined by the presence of a hydrophobic cavity and can form inclusion compounds with other organic molecules, with salts, and with halogens either in solid state or in aqueous solution. Methods for preparing cyclodextrins are well known to those of skill in the art and many cyclodextrins are commercially available.

Cyclodextrins have been utilized in attempts to improve injection site tolerance. For example, WO/0062793 to Vasudevan, et al. discloses methods and compositions for treating fungal infections that include formulations of a pseudomycin or related anti-fungal agent and a cyclodextrin. U.S. Pat. No. 6,048,845 to Rubinfeld discloses compositions of matter including a substituted cyclodextrin and cytotoxic compound. U.S. Pat. No. 5,024,998 to Bodor discloses aqueous parenteral solutions of drugs that are insoluble or only sparingly soluble in water and/or that are unstable in water, combined with hydroxypropyl-62 -cyclodextrin.

Accordingly, there is a need for a composition and method for improving injection site tolerance of a pharmaceutical formulation in the treatment of emesis or improving anesthesia recovery in a subject patient. Further, there is a need for a composition and/or medicament that has improved injection site tolerance for the administration of neurokinin receptor antagonists. Additionally, there is a need for a method of improving injection site tolerance and preventing nausea and emesis and improving anesthesia recovery through the use of a NK-1 antagonist.

### SUMMARY OF INVENTION

In one aspect, the invention is directed to a pharmaceutical composition with an improved injection site toleration comprising an effective amount of a neurokinin receptor (NK-1) antagonist, such as those described in the references cited herein, with a pharmaceutically acceptable cyclodextrin. Further neurokinin receptors are disclosed in U.S. Pat. No. 5,807,867, U.S. Pat. No. 6,222,038, U.S. Pat. No. 6,255,320, U.S. Pat. No. 5,939,433 and U.S. Pat. No. 5,519,033, which are hereby incorporated by reference for all purposes.

In a preferred embodiment, the antagonist is selected from the group consisting of piperazine compounds, spiro-substituted azacycles, dialkyline piperadino compounds, trypthophan urea, polycyclic amine compounds, substituted arylaliphatic compounds, aromatic amine compounds, quaternary ammonium salts or aromatic amine compounds, aryl-substituted heterocycles, polycyclicamine compounds, substituted aryl piperazines, carboxamide derivatives, bis-piperadinyl non-peptidal compounds, salts thereof, and (2S,3S)-2-benzhydryl-N-(5-tert-butyl-2-methoxybenzyl)quinuclidin-3-amine.

In a more preferred embodiment, the antagonist is the compound of Formula Ia, (2S,3S)-2-benzhydryl-N-(5-tert-butyl-2-methoxybenzyl)quinuclidin-3-amine, or a pharmaceutically acceptable salt thereof, preferably the citrate salt, such as the citrate monohydrate salt.

In one embodiment, the cyclodextrin is selected from a pharmaceutically acceptable β-cyclodextrin, hydroxypropyl β-cyclodextrin, sulfobutylether β-cyclodextrin (“SBE-CD”) or substituted cyclodextrins. In another embodiment, the cyclodextrin is about 2% to about 40% of the vehicle by weight. Preferentially, the cyclodextrin is about 4% to about 20% of the composition. More preferably, the cyclodextrin is about 5% to about 10% of the composition and is hydroxypropyl β-cyclodextrin or SBE-CD.

In a preferred embodiment, the therapeutically effective amount of the NK-1 antagonist is about 0.01 mg/kg to about 100 mg/kg of a patient's body weight. More preferably the therapeutically effective amount is about 0.10 mg/kg to about 10 mg/kg.

In another aspect, the invention is directed to a method for the treatment of emesis or improving anesthesia recovery in a mammal using a NK-1 receptor antagonist comprising parenterally injecting into the mammal an aqueous pharmaceutical solution comprising the pharmaceutical composition described above in a therapeutically effective amount sufficient for treating emesis, the cyclodextrin being present in amounts that are sufficient for improving injection toleration at the injection site.

In another aspect, the invention is directed to a method for improving injection site toleration during the treatment of emesis or improving anesthesia recovery in a mammal comprising parenterally injecting into the mammal an aqueous pharmaceutical solution comprising the pharmaceutical composition described above.

In a further aspect, the invention is directed to a pharmaceutical composition, as defined herein, for use as a medicament especially in the treatment of a disease for which a NK-1 receptor antagonist is indicated.

In a further aspect, the invention is directed to the use of a pharmaceutical composition, as defined herein, in the manufacture of a medicament for the treatment of a disease for which a NK-1 receptor antagonist is indicated.

In a further aspect, the invention is directed to a method for the treatment of a disease for which a NK-1 receptor antagonist is indicated in mammals comprising administering to said mammal a therapeutically effective amount of a pharmaceutical composition as defined herein.

### DEFINITIONS

The term(s) “compound(s) of Formula I,” “compound of Formula Ia” and “compound(s) of this invention” as used herein, means a compound or compounds of Formula I or Ia, prodrugs thereof and pharmaceutically acceptable salts of the compounds or the prodrugs. The term “compound(s),” when referring to compounds of Formula Ia, also includes prodrugs of the compound(s) and pharmaceutically acceptable salts of the compound(s) or the prodrugs.

In an embodiment of any of the compositions or methods of the invention, the pharmaceutically acceptable acid may be selected from the group consisting of acetic acid, benzenesulfonic acid, citric acid, hydrobromic acid, hydrochloric acid, D- and L-lactic acid, methanesulfonic acid, phosphoric acid, succinic acid, sulfuric acid, D- and L-tartaric acid, p-toluenesulfonic acid, adipic acid, aspartic acid, camphorsulfonic acid, 1,2-ethanedisulfonic acid, laurylsulfuric acid, glucoheptonic acid, gluconic acid, 3-hydroxy-2-naphthoic acid, 1-hydroxy-2-naphthoic acid, 2-hydroxyethanesulfonic acid, malic acid, mucic acid, nitric acid, naphthalenesulfonic acid, palmitic acid, D-glucaric acid, stearic acid, maleic acid, malonic acid, fumaric acid, benzoic acid, cholic acid, ethanesulfonic acid, glucuronic acid, glutamic acid, hippuric acid, lactobionic acid, lysinic acid, mandelic acid, napadisylic acid, nicotinic acid, polygalacturonic acid, salicylic acid, sulfosalicylic acid, tryptophanic acid, and mixtures thereof. In a preferred embodiment thereof, the acid is citric acid.

The term “citrate salt,” as used herein, refers to the citrate monohydrate salt of the compound of Formula Ia, having a molecular weight of 660.82 and a theoretical potency based on the active ingredient of 709 mg/g.

The term “neurokinin receptor antagonist” as used herein includes, but is not limited to, compounds of Formula I or Ia or various ligands, compounds, and/or substances that can specifically bind to the NK-1 neurokinin receptors and includes, but are not limited to, piperazine compounds, spiro-substituted azacycles, dialkyline piperadino compounds, trypthophan urea, polycyclic amine compounds, substituted arylaliphatic compounds, aromatic amine compounds, quaternary ammonium salts or aromatic amine compounds, aryl substituted hetrocycles, polycyclicamine compounds, substituted aryl piperazines, carboxamide derivatives, bis-piperadinyl non-peptidal compounds, salts thereof, and any other similar neurokinin receptor antagonist known to those of skill in the art. Further neurokinin receptors are those disclosed in U.S. Pat. No. 5,807,867, U.S. Pat. No. 6,222,038, U.S. Pat. No. 6,255,320, U.S. Pat. No. 5,939,433 and U.S. Pat. No. 5,519,033 and are included in the above definition.

The term “cyclodextrin” as used herein means a cyclic oligosaccharide having a hydrophobic interior cavity and a hydrophilic exterior. There are three main types of cyclodextrins: α-cyclodextrin, β-cyclodextrin and γ-cyclodextrin. The term “cyclodextrin” also includes various substituted cyclodextrins, including as side chains any organic moiety or a heteroorganic moiety. Substituted cyclodextrins also include cyclodextrins that have been alkylated, hydroxyalkylated, or reacted to form a sulfoalkyl ether.

As used herein, cyclodextrins and/or substituted cyclodextrins include, but are not limited to, sulfobutylether cyclodextrin, hydroxypropyl cyclodextrin, hydroxyethyl cyclodextrin, glucosyl cyclodextrin, maltosyl cyclodextrin, hydroxypropyl-β-cyclodextrin, sulfobutylether-β-cyclodextrin, hydroxyethyl-β-cyclodextrin, hydroxypropyl-γ-cyclodextrin, hydroxyethyl-β-cyclodextrin, dihydroxypropyl-β-cyclodextrin, glucosyl-β-cyclodextrin, diglycosyl-β-cyclodextrin, maltosyl-β-cyclodextrin, maltosyl-γ-cyclodextrin, maltotrialsyl-β-cyclodextrin, maltotrialsyl-γ-cyclodextrin, dimaltosyl-β-cyclodextrin, cyclodextrin derivatives, various mixtures of cyclodextrin derivatives thereof, mixtures such as maltosyl-β-cyclodextrin/dimaltosyl-β-cyclodextrin, and any other similar cyclodextrin known to those of skill in the art.

The term “pharmaceutically acceptable diluent” is meant to refer to diluents or vehicles, or mixtures thereof, acceptable for parenteral applications in both human and veterinary fields and includes water or other pharmaceutically acceptable excipients for use in making the compositions of the invention (including but not limited to e.g. water for injection, water, water miscible organic solvents, propylene glycol, 2-pyrrolidone, ethanol, n-methyl pyrrolidone, polyethylene glycol, glycerol formal, oily vehicles, sesame oil, safflower oil and the like)

The term “improved injection site toleration” as used herein means a score of two or less, preferably, one or less, in each of the signs of reaction as defined herein in Table 1.

The term “active ingredient” or “mgA/mL”, as used herein, refers to the free base of the compound of Formula Ia, having a molecular weight of 468.69.

The phrase “therapeutically effective amount” means an amount of a compound of the present invention that (i) treats or prevents the particular disease, condition or disorder, (ii) attenuates, ameliorates or eliminates one or more symptoms of the particular disease, condition or disorder, or (iii) prevents or delays the onset of one or more symptoms of the particular disease, condition or disorder described herein.

The phrase “pharmaceutically acceptable” indicates that the substance or composition must be compatible chemically and/or toxicologically, with the other ingredients comprising a formulation, and/or the mammal being treated therewith.

The terms “treating”, “treat” or “treatment” embrace both palliative and preventative (i.e. prophylactic) treatment.

### DESCRIPTION OF INVENTION

The compounds of Formula I or Ia can be prepared as described in U.S. Pat. No. 6,222,038 or U.S. Pat. No. 6,255,038. Salts of the compound of Formula Ia, in particular the citrate salt, can be prepared as described in the above patents or as briefly described below.

For example, the crystalline citrate monohydrate salt of the compound of Formula Ia was prepared by suspending 47 grams of the free base in 470 mL of isopropyl ether under ambient conditions. To the slurry was added 21.42 grams anhydrous citric acid at room temperature. The mixture was converted to the monohydrate by suspending in 150 mL of water for eighteen hours and filtered, providing a white crystalline solid.

With respect to the present invention, formulations are prepared by dissolving a therapeutically effective amount of the compounds of Formula I or Ia in a pharmaceutically acceptable diluent. A pharmaceutically acceptable salt of the compound of Formula Ia may also be used, such as the citrate or malate salts. A cyclodextrin is added to the solution in a concentration range of about 2% to about 40%. Preferably, the cyclodextrin comprises about 4% to about 20% of the pharmaceutical composition and more preferably about 5% to about 10%. Preferably, the cyclodextrin is a β-cyclodextrin: hydroxypropyl β-cyclodextrin, sulfobutylether β-cyclodextrin or other pharmaceutically acceptable substituted β-cyclodextrin.

As used herein, a “therapeutically effective amount” for a dosage unit may typically be about 0.5 mg to about 500 mg of active ingredient. The dose may vary, however, depending on the species, variety, etc. of animal to be treated, the severity and the body weight of the animal. Accordingly, based upon body weight, typical dose ranges of the active ingredient may be from about 0.01 to about 100 mg per kg of body weight of the animal. Preferably, the range is from about 0.10 mg to about 10 mg per kg of body weight.

The veterinary practitioner, or one skilled in the art, will be able to determine the dosage suitable for the particular individual patient, which may vary with the species, age, weight and response of the particular patient. The above dosages are exemplary of the average case. Accordingly, higher or lower dosage ranges may be warranted, depending upon the above factors, and are within the scope of this invention.

Pharmaceutical compositions of the compounds of Formula I or Ia were developed such that a therapeutically effective amount of the compounds of Formula I or Ia could be administered to a patient with an acceptable injection site toleration. Injection site toleration was measured by inspecting the patient for signs of reaction, including erythema (size), skin thickening (size), pain on palpation and edema. Table 1 provides a detailed explanation of the scoring system: a score of 0 (no reaction) to 4 (severe reaction) was given for each characteristic and each injection site daily.

TABLE 1
Explanation of Scoring Systems Used for Subcutaneous Injection Site Toleration

| | | Signs of Reaction | | | |
|---|---|---|---|---|---|
| | Pain on | | Tissue | Pain on | |
| Score | Injection | Erythema | Thickening | Palpation | Edema |
| 0 | no response | no erythema | no | no pain | no edema |
| | | | thickening | | |
| 1 | very slight | Very slight | very slight | Mild pain | very mild |
| | response; | erythema; | reaction; | on deep | edema; |
| | hunch, look at | barely | barely | palpation | barely |
| | site | perceptible | perceptible | | perceptible |
| 2 | mild response; | Mild erythema; | mild, | Mild pain | mild |
| | minor | well defined | palpable | on | palpable |
| | vocalization; | | reaction; ≦1 | palpation | edema |
| | lick/scratch at | | cm | | |
| | site | | | | |
| 3 | moderate | Moderate | moderate, | moderate | moderate |
| | response major | erythema | palpable | pain on | palpable |
| | vocalization; | | reaction | palpation | focal |
| | bite at site, | | 1-2 cm | | edema |
| | motor activity | | | | |
| 4 | severe | Severe | severe | severe | severe |
| | response | erythema | reaction; >2 | pain on | diffuse |
| | similar to 3; >5 | beet redness | cm | palpation | edema |
| | min duration | any eschar | | | |
| | | formation | | | |

The pharmaceutical compositions can further include a preservative to prevent microbial contamination, as more fully described in U.S. Provisional Application, contemporaneously filed, commonly owned and assigned to Pfizer, Inc. The above application is incorporated by reference in its entirety for all purposes. As used herein, the word “preservative” means a compound, or combination of compounds, added to prevent or inhibit the growth of microorganisms which could present a risk of infection or degradation of the medicinal product.

Any of the compositions and/or pharmaceutical compositions described above can be administered solely with the neurokinin receptor antagonist and the cyclodextrin. However, it is possible for additional ingredients to be included within the composition or pharmaceutical composition. Further, various conventional carriers and excipients can be utilized in accord with ordinary practice. Typically, the compositions and/or pharmaceutical compositions are aqueous formulations prepared in sterile form and are isotonic when delivered. Additional excipients include, but are not limited to, antioxidants, chelating agents such as ethylenediaminetetraacetic acid (“EDTA”), carbohydrates, and any other similar ingredients known to those of skill in the art. Furthermore, the apparent pH of the formulations ranges from about three to about seven, but is ordinarily from about four to about six. With regard to the various carriers, any known pharmaceutically acceptable carrier, that properly solubilizes the NK-1 antagonist can be utilized with the present invention.

The compositions and pharmaceutical compositions of the present invention can be administered in a number of ways; most preferably parenterally.

### GENERAL EXPERIMENTAL PROCEDURES

Unless specified otherwise, commercial reagents were utilized without further purification and may be obtained from, for example, Sigma or Aldrich. HPB-CD (Cavitron 82003) was obtained from Cargill. Ethyl oleate (Crodamol) was obtained from Croda Inc. Miglyol 812 (Nutralol) was obtained from Condia.

Individual sodium chloride, calcium choride, and sodium acetate 1% solutions were prepared by dissolving 1 gram of the respective salt in sufficient water for injection to provide a final volume of 100 mL. One skilled in the art would appreciate that alternate volumes of solution may be prepared by scaling the volume of the solution components as appropriate in relation to the amount of salt added.

Forty (40)% glycerol formal solutions were prepared by dispersing 40 grams glycerol formal in sufficient water for injection to produce a final volume of 100 mL.

The following Examples are intended to illustrate particular embodiments of the invention and are not intended to limit the specification, including the claims in any manner.

### EXAMPLE

### Injection Site Toleration Study of Compound of Formula Ia

The injection site toleration of compound of Formula Ia in various pharmaceutically acceptable diluents was evaluated. The compound of Formula Ia was administered by subcutaneous injection to beagle or mongrels dogs at 1 mg/kg/day for one to four consecutive days. Dogs were observed immediately following each dose for evidence of pain on injection. All injection sites were evaluated daily until at least twenty-four hours after the last injection for evidence of reaction.

The following formulations utilized in the injection site toleration study were prepared as described below. The formulations provide the final concentration of the active ingredient, the compound of Formula Ia, prepared from the citrate salt of the compound of Formula Ia, having an actual potency of 692 mg/g, unless designated otherwise.

The formulation solutions were filtered through a 0.22 micron Millipore GV filter membrane into sterilized 30 mL vial(s) closed with a rubber stopper, except for Examples Y, Z, AA, BB, CC, DD, EE and II that were filtered through a 0.45 micron Millipore HV filter membrane into a sterilized 20 mL vial(s) closed with a rubber stopper.

For those Examples having sulfobutylether β-cyclodextrin (“SBE-CD”) as part of the pharmaceutical composition, the sodium salt of SBE-CD was utilized.

### Example A

### 1% Sodium Chloride; 10 mg/mL Compound of Formula Ia

A 10 mg/mL solution of compound of Formula Ia was prepared by dissolving 0.51 grams of the citrate salt of the Compound of Formula Ia in 34.49 grams of a 1% sodium chloride solution, providing approximately 35 mL of solution with a pH of 3.89.

### Example B

### 1% Calcium Chloride; 10 mg/mL Compound of Formula Ia

A 10 mg/mL solution of compound of Formula Ia was prepared by dissolving 0.51 grams of the citrate salt of the compound of Formula Ia in 34.51 grams of a 1% calcium chloride solution, providing approximately 35 mL of solution with a pH of 3.45.

### Example C

### 1% Sodium Acetate; 10 mg/mL Compound of Formula Ia

A 10 mg/mL solution of compound of Formula Ia was prepared by dissolving 0.51 grams of the citrate salt of the compound of Formula Ia in 34.51 grams of a 1% sodium acetate solution, providing approximately 35 mL of solution with a pH of 5.24.

### Example D

### 40% Glycerol Formal; 10 mg/mL Compound of Formula Ia

A 10 mg/mL solution of compound of Formula Ia was prepared by dissolving 0.51 grams of the citrate salt of the compound of Formula Ia in 37.78 grams of a 40% glycerol formal solution, providing approximately 35 mL of solution with an apparent pH of 4.55.

### Example E

### 25% 2-pyrrolidone; 10 mg/mL Compound of Formula Ia

A 10 mg/mL solution of compound of Formula Ia was prepared by adding 0.51 grams of the citrate salt of the compound of Formula Ia to 36.30 grams of a 25% 2-pyrrolidone solution (25 grams 2-pyrrolidone in sufficient water for injection (78.27 grams) to make 100 mL of solution). To enhance dissolution of the compound of Formula Ia, 10% hydrochloric acid (“HCl”) (6.75 grams of concentrated HCl in sufficient water for injection (18.24 grams) to give 25.00 grams of solution) was added in portions of 5, 5, 10, 10, 10, 50, and 50 μL for a total of 140 μL, providing approximately 35 mL of solution with an apparent pH of 4.05.

### Example F

### 1% Calcium Chloride; 5 mg/mL Compound of Formula Ia

A 5 mg/mL solution of Compound of Formula Ia was prepared by dissolving 0.51 grams of the citrate salt of Compound of Formula Ia in 69.49 grams of a 1% calcium chloride solution, providing approximately 70 mL of solution with a pH of 3.54.

### Example G

### 1% Calcium Chloride: 10 mg/mL Compound of Formula Ia

A 10 mg/mL solution of Compound of Formula Ia was prepared by dissolving 0.51 grams of the citrate salt of Compound of Formula Ia in 34.50 grams of a 1% calcium chloride solution, providing approximately 35 mL of solution with a pH of 3.45.

### Example H

### 40% Glycerol Formal; 5 mg/mL Compound of Formula Ia

A 5 mg/mL solution of Compound of Formula Ia was prepared by dissolving 0.51 grams of the citrate salt of compound of Formula Ia in 75.09 grams of a 40% glycerol formal solution, providing approximately 70 mL of solution with an apparent pH of 4.64.

### Example I

### 40% Glycerol Formal, 10 mg/mL Compound of Formula Ia

A 10 mg/mL solution of compound of Formula Ia was prepared by dissolving 0.51 grams of the citrate salt of compound of Formula Ia in 37.29 grams of a 40% glycerol formal solution, providing approximately 35 mL of solution with an apparent pH of 4.56.

### Example J

### 20% SBE-CD; 10 mg/mL Compound of Formula Ia

A 10 mg/mL solution of compound of Formula Ia was prepared by dissolving 1.45 grams of the citrate salt of the compound of Formula Ia in sufficient 20% SBE-CD solution (20 grams of SBE-CD dissolved in sufficient water for injection to produce volume of 100 mL).

### Example K

### 1% Calcium Chloride; 10 mg/mL Compound of Formula Ia

A 10 mg/mL solution of compound of Formula Ia was prepared by dissolving 0.51 grams of the citrate salt of Compound of Formula Ia in 33.89 grams of a 1% calcium chloride/sodium hydroxide solution (0.52 grams of a 10% sodium hydroxide solution (2.50 grams of sodium hydroxide dissolved in sufficient water for injection to make 25.00 grams of solution) was added to a 1% calcium chloride solution), providing approximately 35 mL of solution with a pH of 5.00.

### Example L

### 1% Calcium Chloride; 10 mg/mL Compound of Formula Ia

A 10 mg/mL solution of compound of Formula Ia was prepared by dissolving 0.45 grams of the malate salt of compound of Formula Ia (theoretical potency 780 mg/gram) in 34.58 grams of a 1% calcium chloride solution, providing approximately 35 mL of solution with a pH of 3.76.

### Example M

### 40% Glycerol Formal/Phosphate Buffer; 10 mg/mL Compound of Formula Ia

A 100 millimolar solution of sodium dihydrogen phosphate dihydrate (“NaH₂PO₄.2H₂O”) was prepared by dissolving 1.38 grams of NaH₂PO₄.2H₂O in sufficient water for injection to make 100 mL of solution. A 100 millimolar solution of phosphoric acid (“H₃PO₄”) was prepared by dispersing 1.13 grams 86.7% H₃PO₄ in sufficient water for injection to make 100 mL of solution. A 100 millimolar pH 2.02 phosphate buffer was prepared by combining 60 mL of the NaH₂PO₄.2H₂O solution whose preparation is described above and 45 mL of the H₃PO₄ solution whose preparation is described above. A 40% (weight/volume) solution of glycerol formal in 50 millimolar phosphate buffer was prepared by dispersing 40.15 grams of glycerol formal in 49.0 grams of the 100 millimolar, pH 2 phosphate buffer and sufficient water for injection (19.47 grams) to make 100 mL of solution. The apparent pH of the resulting solution was 2.61.

A 10 mg/mL solution of compound of Formula Ia was prepared by dissolving 0.504 grams of the citrate salt of Compound of Formula Ia in 38.06 grams of the 40% glycerol formal solution whose preparation is described above. The pH was adjusted by adding 10% HCl (13.5 grams of concentrated HCl in sufficient water for injection to give 50 grams of solution) in portions of 20, 50, 50, 40, and 20 μL for a total of 180 μL, providing approximately 36 mL of solution with an apparent pH of 3.01.

### Example N

### 25% N-methylpyrrolidone: 10 mg/mL Compound of Formula Ia

A 10 mg/mL solution of Compound of Formula Ia was prepared by adding 0.510 grams of the citrate salt of compound of Formula Ia to 35.44 grams of a 25% N-methylpyrrolidone (“NMP”) solution (12.51 grams of N-methylpyrrolidone in sufficient water for injection (38.08 grams) to make 50 mL of solution), providing approximately 36 mL of solution with an apparent pH of of 4.60.

### Example O

### 1% Calcium Chloride: 10 mg/mL Compound of Formula Ia

A 10 mg/mL solution of compound of Formula Ia was prepared by dissolving 0.35 grams of the free base of compound of Formula Ia (theoretical potency 1000 mg/gram) in 34.30 grams of a 1% calcium chloride solution to which was added 0.30 grams of 10% HCl (13.5 grams of concentrated was dispersed in sufficient water for injection to give 50 grams of solution), providing approximately 35 mL of solution with a pH of 4.10.

### Example P

### 5% SBE-CD; 10 mg/mL Compound of Formula Ia

A 10 mg/mL solution of compound of Formula Ia was prepared by adding 0.504 grams of the citrate salt of compound of Formula Ia to 35.60 grams of a 5% SBE-CD solution (5.00 grams of the sodium salt of SBE-CD dissolved in sufficient water for injection (96.73 grams) to make 100 mL), providing approximately 35 mL of solution with a pH of 4.46.

### Example Q

### 5% SBE-CD/1% Calcium Chloride; 10 mg/mL Compound of Formula Ia

A solution containing 5% SBE-CD and 1% calcium chloride was prepared by dissolving 0.3 grams of calcium chloride in 30.7 grams of the 5% SBE-CD (preparation described above) to give approximately 30 mL of solution. A 10 mg/mL solution of compound of Formula Ia was prepared by adding 0.44 grams of the citrate salt of compound of Formula Ia to 30.7 grams of the 5% SBE-CD/1% calcium chloride solution, providing approximately 31 mL of solution with a pH of 4.55.

### Example R

### 30% PEG400; 10 mg/mL Compound of Formula Ia

A 10 mg/mL solution of compound of Formula Ia was prepared by dispersing 2.67 grams of the citrate salt of compound of Formula Ia in 191.99 grams of 30% polyethylene glycol 400 (“PEG-400”) solution (90.06 grams of PEG400 in sufficient water for injection (223.17 grams) to make 300 mL of solution). The pH was adjusted by adding 10% HCl (13.5 grams of concentrated (37% weight/weight) HCl dispersed in sufficient water for injection to give 50 grams of solution) in portions of 1.98 grams and 0.407 grams for a total of 2.39 grams, providing approximately 189 mL of final solution with an apparent pH of 2.97.

### Example S

### 30% PG; 10 mg/mL Compound of Formula Ia

A 10 mg/mL solution of compound of Formula Ia was prepared by dispersing 2.76 grams of the citrate salt of compound of Formula Ia in 193.33 grams of a 30% propylene glycol (“PG”) solution (90.01 grams of PG dispersed in sufficient water for injection (218.53 grams) to make 300 mL of solution). The pH was adjusted by adding 10% HCl in portions of 1.88 grams and 0.39 grams for a total of 2.27 grams, providing approximately 193 mL of final solution with an apparent pH of 3.01.

### Example T

### 1% Calcium Chloride; 10 mg/mL Compound of Formula Ia

A 10 mg/mL solution of compound of Formula Ia was prepared by dissolving 0.35 grams of the free base of compound of Formula Ia (theoretical potency 1000 mg/grams) in 33.90 grams of a 1% calcium chloride solution to which was added 0.76 grams of a 10% methanesulfonic acid solution (1 gram of methanesulfonic acid and 9 grams of water for injection to give 10 grams of solution), providing approximately 35 mL of solution with a pH of 4.17. (The molar concentration of methanesulfonic acid was slightly greater than the molar concentration of the compound of Formula Ia.)

### Example U

### Water for Injection; 10 mg/mL Compound of Formula Ia

A 10 mg/mL solution of compound of Formula Ia was prepared by dissolving 0.35 grams of the free base of compound of Formula Ia (theoretical potency 1000 mg/grams) in 33.91 grams in water for injection to which was added 0.87 grams of a 10% methanesulfonic acid solution, providing approximately 35 mL of solution with a pH of 4.07.

### Example V

### 1.3% Calcium Chloride; 10 mg/mL Compound of Formula Ia

A 10 mg/mL solution of compound of Formula Ia was prepared by adding 0.51 grams of the citrate salt of compound of Formula Ia to 34.50 grams of the 1.3% calcium chloride solution (1.3 grams of calcium chloride was dissolved in sufficient water for injection to make 100 mL of solution), providing approximately 35 mL of solution with a pH of 3.52.

### Example W

### 10% HPB-CD; 10 mg/mL Compound of Formula Ia

A 10 mg/mL solution of compound of Formula Ia was prepared by dissolving 2.88 grams of the citrate salt of compound of Formula Ia in 203.99 grams of a 10% hydroxypropyl R-cyclodextrin (“HPB-CD”) solution (30.97 grams of HPB-CD dissolved in sufficient water for injection (213.62 grams) to make 300 mL of solution). The pH was adjusted by adding 0.44 grams of a 10% NaOH (10 grams of NaOH in sufficient water for injection to give 100 mL) and 0.066 grams of a 10% HCl solution, providing approximately 202 mL of solution with a pH of 4.40.

### Example X

### 10% SBE-CD; 10 mg/mL Compound of Formula Ia

A 10 mg/mL solution of compound of Formula Ia was prepared by dissolving 1.45 grams of the citrate salt of compound of Formula Ia in a sufficient amount of a 10% SBE-CD solution (10 grams of SBE-CD dissolved in sufficient water to make 100 mL of solution) to provide 100 mL of solution.

### Example Y

### 75% Sesame Oil/25% Ethyl Oleate; 10 mg/mL Compound of Formula Ia

A 10 mg/mL solution of compound of Formula Ia in 3:1 (volume/volume) sesame oil:ethyl oleate was prepared by dissolving 0.166 grams of the free base of compound of Formula Ia (theoretical potency 1000 mg/gram) in 11.87 grams (12.75 mL) of sesame oil and 3.59 grams (4.25 mL) of ethyl oleate, providing approximately 17 mL of solution.

### Example Z

### Miglyol; 10 mg/mL Compound of Formula Ia

A 10 mg/mL solution of compound of Formula Ia in Miglyol 812 was prepared by dissolving 0.17 grams of the free base of the compound of Formula Ia (theoretical potency 1000 mg/gram) in 15.90 grams (17 mL) of Miglyol 812, providing approximately 17 mL of solution.

### Example AA

### 75% Safflower Oil/25% Ethyl Oleate; 10 mg/mL Compound of Formula Ia

A 10 mg/mL solution of compound of Formula Ia in 3:1 (volume/volume) safflower oil:ethyl oleate was prepared by dissolving 0.177 grams of the free base of the compound of Formula Ia (theoretical potency 1000 mg/gram) in 11.81 grams (12.75 mL) of safflower oil and 3.60 grams (4.25 mL) of ethyl, providing approximately 17 mL of solution.

### Example BB

### Micellar; 10 mg/mL Compound of Formula Ia

A 10 mg/mL solution of compound of Formula Ia was prepared by charging a glass vessel with 13.01 grams of water for injection and adding 0.55 grams of a 10 molar sodium hydroxide solution (200.04 grams of NaOH dissolved water for injection to a final volume of 500 mL) and 2.21 grams of glycocholic acid with stirring until the acid dissolved. The solution was heated to 50° C. 4.23 grams of lecithin and 3.75 grams of an arginine solution (0.752 grams of arginine dissolved in 3.02 grams of water for injection) were added and the solution held at 50° C. To this was added 0.36 grams of the citrate salt of compound of Formula Ia and the pH was adjusted by addition of 1.24 grams of a 10% HCl and 0.55 grams of a 1 molar sodium hydroxide (20.07 grams of NaOH dissolved in water for injection for final volume of 500 mL), providing approximately 25 mL of solution with a pH of 6.5.

### Example CC

### 12.5% Cremaphor/12.5% Ethanol/75% Saline; 10 mg/mL Compound of Formula Ia

A 10 mg/mL solution of compound of Formula Ia was prepared by dissolving 0.51 grams of the citrate salt of the compound of Formula Ia in 8.75 grams of a 50% Cremophor in ethanol solution (50 grams of Cremophor EL (BASF) dissolved in ethanol (dehydrated, 200 proof)) for final volume of 100 mL) and 25.50 grams of commercial 0.9% saline, providing approximately 35 mL of solution with an apparent pH of 4.27.

### Example DD

### 25% Cremaphor/25% Ethanol/50% Saline; 10 mg/mL Compound of Formula Ia

A 10 mg/mL solution of compound of Formula Ia was prepared by dissolving 0.51 grams of the citrate salt of compound of Formula Ia in 17.54 grams of a 50% Cremophor in ethanol solution and 16.25 grams of commercial 0.9% saline, providing approximately 35 mL of solution with an apparent pH of 4.90.

### Example EE

### 40% Ethyl Oleate in Sesame Oil; 10 mg/mL Compound of Formula Ia

A 10 mg/mL solution of compound of Formula Ia was prepared by dissolving 0.26 grams of the free base of compound of Formula Ia (theoretical potency 1000 mg/g) in 23.26 grams of the 40% ethyl oleate in sesame oil vehicle (20.01 grams of ethyl oleate in 24.72 grams sesame oil to make 50 mL), providing approximately 25 mL of solution.

### Example FF

### 5% SBE-CD/1 % Sodium Acetate; 10 mg/mL Compound of Formula Ia

A 10 mg/mL solution of compound of Formula Ia was prepared by dissolving 0.43 grams of the citrate salt of compound of Formula Ia in 29.90 grams of a 1% sodium acetate/5% SBE-CD solution (1 grams of sodium acetate and 5 grams of the sodium salt of SBE CD dissolved in water for injection for a final volume of 100 mL), providing approximately 30 mL of solution with a pH of 5.18.

### Example GG

### 5% SBE-CD/25% PG; 10 mg/mL Compound of Formula Ia

A 10 mg/mL solution of compound of Formula Ia was prepared by dissolving 0.43 grams of the citrate salt of compound of Formula Ia in 30.70 grams of a 5% SBE-CD/25% PG solution (5 grams of the sodium salt of SBE-CD and 25 grams of PG dissolved in water for injection for a final volume of 100 mL), providing approximately 30 mL of solution with an apparent pH of 4.53.

### Example HH

### 5% SBE-CD/25% NMP: 10 mg/mL Compound of Formula Ia

A 10 mg/mL solution of compound of Formula Ia was prepared by dissolving 0.362 grams of the citrate salt of the compound of Formula Ia in 25.82 grams of a 25% of N-methylpyrrolidone/5% SBE-CD solution (2.52 grams of the sodium salt of SBE-CD and 12.50 grams of N-methylpyrrolidone (“NMP”)(Acros) dissolved in water for injection (36.57 g) for a final volume of 50 mL), providing approximately 25 mL of solution with an apparent pH of 4.73.

### Example II

### 50% Ethyl Oleate in Sesame Oil; 10 mg/mL Compound of Formula Ia

A 10 mg/mL solution of compound of Formula Ia was prepared by dissolving 0.259 grams of the free base of compound of Formula Ia (theoretical potency 1000 mg/g) in 23.04 grams of a 50% ethyl oleate in sesame oil vehicle (25.02 grams of ethyl oleate dispersed in 19.47 grams sesame oil for a final volume of 50 mL), providing approximately 25 mL of solution.

### Example JJ

### 10% SBE-CD/25% PG; 10 mg/mL Compound of Formula Ia

A 10 mg/mL solution of compound of Formula Ia was prepared by dissolving 0.43 grams of the citrate salt of compound of Formula Ia in 31.16 grams of a 10% SBE-CD/25% PG solution (10 grams of the sodium salt of SBE CD and 25 grams of PG dissolved in water for injection for a final volume of 100 mL), providing approximately 30 mL of solution with an apparent pH of 4.47.

### Example KK

### 10% SBE-CD; 10 mg/mL Compound of Formula Ia

A 10 mg/mL solution of compound of Formula Ia was prepared by dissolving 0.38 grams of the malate salt of compound of Formula Ia (theoretical potency 780 mg/gram) in 30.70 grams of a 10% SBE-CD solution, providing approximately 30 mL of solution with a pH 4.55.

### Example LL

### 10% SBE-CD: 10 mg/mL Compound of Formula Ia

A 10 mg/mL solution of compound of Formula Ia was prepared by dissolving 0.434 grams of the citrate salt of compound of Formula Ia in 31.25 grams of a 10% SBE-CD solution. The pH was adjusted by adding 0.38 grams of 10% HCl and 0.04 grams of 10% NaOH, providing approximately 30 mL of solution with a pH of 3.02.

### Example MM

### 7.5% SBE-CD; 10 mg/mL Compound of Formula Ia

A 10 mg/mL solution of the citrate salt of the compound of Formula Ia containing 7.5% SBE-CD was prepared as follows. Water for injection (13175 g) was charged into a glass-lined carboy. The water was heated to 30-40° C. and maintained in this temperature range during compounding. SBE-CD (1313 g) was added to the carboy and stirred until dissolved. The citrate salt of the compound of Formula Ia (252 g) was added to the carboy and stirred until dissolved. An additional portion of water for injection (3295 g) was added to the carboy and stirred until dispersed. The solution was cooled to 20-30° C., producing approximately 17500 mL of solution containing 10 mg/mL of the compound of Formula Ia and 7.5% (weight/volume) SBE-CD with a pH of 4.4.

The resulting solution was filtered through redundant Millipore 0.2 micron KVGL04TC3 sterilizing filters into a sterilized glass-lined receiving tank. A portion of the solution was filled into 20 mL amber glass vials in an aseptic processing area. The vial headspace was flushed with filtered nitrogen, and the vials were closed and sealed with rubber stoppers and aluminum crimps. The vials were placed in an autoclave and heated to 121° C., held at that temperature for approximately 15 minutes, and cooled to room temperature.

The above-described formulations were subcutaneously injected as described above. Table 2 compiles the formulation descriptions and mean injection site toleration scores.

TABLE 2
Formulation Details And Mean Injection Site Toleration Scores

| | | Compound of | | | Mean IST Scores | | | | | | |
|---|---|---|---|---|---|---|---|---|---|---|---|
| | | Form. I Conc. | | | Pain on | | Size | Skin | Size | Pain on | |
| Vehicle | Example | (mg/mL) | # days | # sites | Injection | Erythema | (cm²) | Thickening | (cm²) | Palpation | Edema |
| 1% Sodium | A | 10 | 4 | 1 | 0.5 | 0.3 | 1.3 | 1.5 | 5.2 | 0 | 0.1 |
| Chloride | | | | | | | | | | | |
| 1% Calcium | B | 10 | 4 | 1 | 0.2 | 0 | 0 | 1.0 | 3.5 | 0.1 | 0.3 |
| Chloride | | | | | | | | | | | |
| 1% Sodium | C | 10 | 4 | 1 | 0.8 | 0.1 | 0.3 | 1.6 | 6.7 | 0.2 | 0.3 |
| Acetate | | | | | | | | | | | |
| 40% Glycerol | D | 10 | 4 | 1 | 0.3 | 0.3 | 0.9 | 1.4 | 6.7 | 0.1 | 0.5 |
| Formal | | | | | | | | | | | |
| 25% 2- | E | 10 | 4 | 1 | 0.2 | 0 | 0 | 1.9 | 7.7 | 0.1 | 0.3 |
| pyrrolidone | | | | | | | | | | | |
| 1% Calcium | F | 5 | 3 | 1 | 0.1 | 0 | 0 | 0.8 | 5.3 | 0 | 0 |
| Chloride | | | | | | | | | | | |
| 1% Calcium | G | 10 | 4 | 4 | 0.3 | 0 | 0 | 0.9 | 3.7 | 0 | 0 |
| Chloride | | | | | | | | | | | |
| 40% Glycerol | H | 5 | 3 | 1 | 0.5 | 0 | 0 | 1.4 | 9.0 | 0 | 0.4 |
| Formal | | | | | | | | | | | |
| 40% Glycerol | I | 10 | 4 | 4 | 0.6 | 0 | 0 | 0.8 | 2.9 | 0 | 0.1 |
| Formal | | | | | | | | | | | |
| 20% SBE | J | 10 | 4 | 1 | 0.1 | 0 | 0 | 0 | 0 | 0 | 0 |
| Cyclodextrin | | | | | | | | | | | |
| 20% SBE | J | 10 | 4 | 1 | 0 | 0 | 0 | 0 | 0 | 0 | 0 |
| Cyclodextrin | | | | | | | | | | | |
| 1% Calcium | K | 10 | 1 | 1 | 0 | 0 | 0 | 0.8 | 1.6 | 0 | 0.1 |
| Chloride | | | | | | | | | | | |
| pH 5.0 | | | | | | | | | | | |
| 1% Calcium | K | 10 | 3 | 1 | 0 | 0 | 0 | 1.6 | 5.4 | 0 | 0.3 |
| Chloride | | | | | | | | | | | |
| pH 5.0 | | | | | | | | | | | |
| 1% Calcium | L | 10 | 1 | 1 | 0 | 0 | 0 | 0.7 | 1.3 | 0 | 0 |
| Chloride | | | | | | | | | | | |
| pH 3.8 | | | | | | | | | | | |
| 1% Calcium | L | 10 | 3 | 1 | 0 | 0 | 0 | 1.5 | 6.3 | 0 | 0.1 |
| Chloride | | | | | | | | | | | |
| pH 3.8 | | | | | | | | | | | |
| 40% Glycerol | M | 10 | 1 | 1 | 0.8 | 0 | 0 | 0.5 | 1.2 | 0 | 0 |
| Formal, | | | | | | | | | | | |
| Phosphate | | | | | | | | | | | |
| Buffer | | | | | | | | | | | |
| pH 3.0 | | | | | | | | | | | |
| 40% Glycerol | M | 10 | 3 | 1 | 0.3 | 0 | 0 | 1.3 | 5.9 | 0 | 0.4 |
| Formal | | | | | | | | | | | |
| pH 3.0 | | | | | | | | | | | |
| 25% NMP | N | 10 | 1 | 1 | 1.5 | 0 | 0 | 0.3 | 0.7 | 0 | 0 |
| pH 4.6 | | | | | | | | | | | |
| 25% NMP | N | 10 | 3 | 1 | 0 | 0.2 | 0.3 | 1.3 | 4.9 | 0 | 0 |
| pH 4.6 | | | | | | | | | | | |
| 1% Calcium | O | 10 | 1 | 1 | 0 | 0 | 0 | 0.8 | 2.8 | 0 | 0 |
| Chloride | | | | | | | | | | | |
| pH 4.1 | | | | | | | | | | | |
| 1% Calcium | O | 10 | 3 | 1 | 0.2 | 0 | 0 | 1.6 | 6.6 | 0 | 0.1 |
| Chloride | | | | | | | | | | | |
| pH 4.1 | | | | | | | | | | | |
| 5% SBE | P | 10 | 1 | 1 | 1.0 | 0 | 0 | 0 | 0 | 0 | 0 |
| Cyclodextrin | | | | | | | | | | | |
| pH 4.5 | | | | | | | | | | | |
| 5% SBE | P | 10 | 4 | 1 | 0.1 | 0 | 0 | 0 | 0 | 0 | 0 |
| Cyclodextrin | | | | | | | | | | | |
| pH 4.5 | | | | | | | | | | | |
| 1% Calcium | Q | 10 | 1 | 1 | 0.3 | 0 | 0 | 0 | 0 | 0 | 0 |
| Chloride/ | | | | | | | | | | | |
| 5% SBE -CD | | | | | | | | | | | |
| 1% Calcium | Q | 10 | 4 | 1 | 0 | 0 | 0 | 0 | 0 | 0 | 0 |
| Chloride/ | | | | | | | | | | | |
| 5% SBE-CD | | | | | | | | | | | |
| 30% PEG- | R | 10 | 1 | 1 | 1.0 | 0 | 0 | 0.4 | 0.5 | 0 | 0 |
| 400 | | | | | | | | | | | |
| 30% PEG- | R | 10 | 3 | 1 | 0.3 | 0 | 0 | 1.6 | 3.4 | 0 | 0.3 |
| 400 | | | | | | | | | | | |
| 30% PG | S | 10 | 1 | 1 | 1.5 | 0 | 0 | 0.4 | 0.8 | 0 | 0 |
| 30% PG | S | 10 | 3 | 1 | 0.3 | 0 | 0 | 2.3 | 5.1 | 0 | 0 |
| 1% Calcium | T | 10 | 1 | 1 | 0 | 0.5 | 0.9 | 1.1 | 2.7 | 0 | 0 |
| Chloride | | | | | | | | | | | |
| 1% Calcium | T | 10 | 3 | 1 | 0.2 | 0.7 | 1.8 | 2.0 | 5.8 | 0 | 0 |
| Chloride | | | | | | | | | | | |
| Water for | U | 10 | 1 | 1 | 0.8 | 0 | 0 | 0.8 | 1.1 | 0 | 0 |
| injection | | | | | | | | | | | |
| Water for | U | 10 | 3 | 1 | 0.3 | 0.6 | 2.0 | 2.7 | 6.2 | 0 | 0 |
| injection | | | | | | | | | | | |
| 1.3% Calcium | V | 10 | 1 | 1 | 0.3 | 0 | 0 | 1.3 | 2.3 | 0 | 0 |
| Chloride | | | | | | | | | | | |
| (isotonic) | | | | | | | | | | | |
| 1.3% Calcium | V | 10 | 3 | 1 | 0.2 | 0 | 0 | 2.4 | 6.5 | 0 | 0.1 |
| Chloride | | | | | | | | | | | |
| (isotonic) | | | | | | | | | | | |
| 10% HPB- | W | 10 | 1 | 1 | 0.5 | 0 | 0 | 0.1 | 0.1 | 0 | 0 |
| CD | | | | | | | | | | | |
| 10% HPB-CD | W | 10 | 4 | 1 | 0.1 | 0 | 0 | 0.1 | 0.3 | 0 | 0 |
| 10% SBE-CD | X | 10 | 1 | 1 | 0 | 0 | 0 | 0 | 0 | 0 | 0 |
| 10% SBE-CD | X | 10 | 4 | 1 | 0 | 0 | 0 | 0 | 0 | 0 | 0 |
| 75% Sesame | Y | 10 | 4 | 1 | 0.2 | 0 | 0 | 1.0 | 3.1 | 0 | 0.3 |
| oil/25% Ethyl | | | | | | | | | | | |
| Oleate | | | | | | | | | | | |
| Miglyol 812 | Z | 10 | 3 | 1 | 0.3 | 0 | 0 | 2.7 | 21 | 0.2 | 1.5 |
| 75% Safflower | AA | 10 | 3 | 1 | 0.1 | 0 | 0 | 1.9 | 12 | 0 | 0.9 |
| Oil/25% | | | | | | | | | | | |
| Ethyl Oleate | | | | | | | | | | | |
| Micellar | BB | 10 | 4 | 1 | 0 | 0 | 0 | 1.1 | 2.5 | 0 | 0 |
| 12.5% | CC | 10 | 4 | 1 | 1.0 | 0 | 0 | 1.5 | 3.3 | 0.4 | 0 |
| cremaphor, | | | | | | | | | | | |
| 12.5% | | | | | | | | | | | |
| ethanol, 75% | | | | | | | | | | | |
| saline | | | | | | | | | | | |
| 25% | DD | 10 | 4 | 1 | 0 | 0 | 0 | 1.3 | 2.7 | 0 | 0 |
| cremaphor, | | | | | | | | | | | |
| 25% ethanol, | | | | | | | | | | | |
| 50% saline | | | | | | | | | | | |
| 40% Ethyl | EE | 10 | 4 | 1 | 0 | 0 | 0 | 1.4 | 1.4 | 0 | 0.1 |
| Oleate in | | | | | | | | | | | |
| Sesame Oil | | | | | | | | | | | |
| 5% SBE-CD, | FF | 10 | 4 | 1 | 0.1 | 0 | 0 | 0.3 | 0.5 | 0 | 0 |
| 1% sodium | | | | | | | | | | | |
| acetate | | | | | | | | | | | |
| 5% SBE-CD, | GG | 10 | 4 | 1 | 0 | 0 | 0 | 0.9 | 2.3 | 0 | 0 |
| 25% PG | | | | | | | | | | | |
| 5% SBE -CD, | HH | 10 | 4 | 1 | 0.6 | 0 | 0 | 1.8 | 3.8 | 0 | 0 |
| 25% NMP | | | | | | | | | | | |
| 50% Ethyl | II | 10 | 4 | 1 | 0.1 | 0 | 0 | 0.6 | 1.3 | 0 | 0 |
| Oleate in | | | | | | | | | | | |
| Sesame oil | | | | | | | | | | | |
| 10% SBE- | JJ | 10 | 4 | 1 | 0.3 | 0 | 0 | 0.8 | 0.8 | 0 | 0 |
| CD, 25% PG | | | | | | | | | | | |
| 10% SBE-CD | KK | 10 | 4 | 1 | 0 | 0 | 0 | 0.1 | 0.1 | 0 | 0 |
| 10% SBE-CD | LL | 10 | 4 | 1 | 0.1 | 0 | 0 | 0 | 0 | 0 | 0 |

### Preferred Embodiments

1. A pharmaceutical composition with an improved injection site toleration comprising a therapeutically effective amount of a neurokinin receptor (NK-1) antagonist with a pharmaceutically acceptable cyclodextrin.

2. A pharmaceutical composition according to preferred embodiment 1 wherein the antagonist is selected from the group consisting of piperazine compounds, spiro-substituted azacycles, dialkyline piperadino compounds, trypthophan urea, polycyclic amine compounds, substituted arylaliphatic compounds, aromatic amine compounds, quaternary ammonium salts or aromatic amine compounds, aryl-substituted heterocycles, polycyclicamine compounds, substituted aryl piperazines, carboxamide derivatives, and bis-piperadinyl non-peptidal compounds, or salts thereof.

3. The pharmaceutical composition of Preferred embodiment 1 wherein the NK-1 antagonist, is a compound of Formula I, 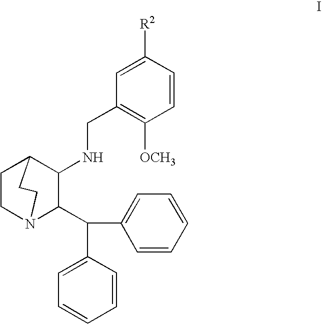
or pharmaceutically acceptable salt or prodrug thereof, wherein R² is selected from the group consisting of methyl, ethyl, isopropyl, sec-butyl and tert-butyl with a pharmaceutically acceptable cyclodextrin.

4. A pharmaceutical composition according to preferred embodiment 3 wherein the compound of Formula I is a compound of Formula Ia, 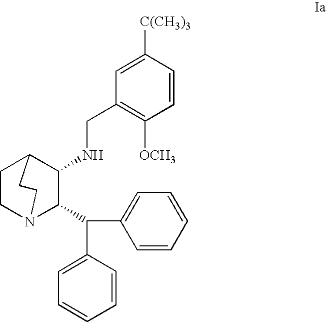
or a pharmaceutically acceptable salt or prodrug thereof.

5. The pharmaceutical composition according to Preferred embodiments 1, 2, 3 or 4 wherein the cyclodextrin is selected from β-cyclodextrin, hydroxypropyl β-cyclodextrin, sulfobutylether β-cyclodextrin or substituted cyclodextrins.

6. The pharmaceutical composition according to Preferred embodiment 5 wherein the cyclodextrin is about 2% to about 40% of the composition.

7. The pharmaceutical composition according to Preferred embodiment 6 wherein the cyclodextrin is about 4% to about 20% of the composition.

8. A pharmaceutical composition according to Preferred embodiment 7 wherein the cyclodextrin is about 5% to about 10% of the composition.

9. A pharmaceutical composition according to Preferred embodiment 8 wherein the cyclodextrin is sulfobutylether β-cyclodextrin or hydroxypropyl β-cyclodextrin.

10. A pharmaceutical composition according to Preferred embodiment 9 wherein the therapeutically effective amount of the NK-1 antagonist is 0.01 mg/kg to 100 mg/kg of a patient's body weight.

11. A pharmaceutical composition according to Preferred embodiment 10 wherein the therapeutically effective amount is 0.10 mg/kg to 10 mg/kg of a patient's body weight.

12. The pharmaceutical composition according to Preferred embodiment 11 wherein the pharmaceutically acceptable salt is citrate.

13. A method for the treatment of emesis or improving anesthesia recovery in a mammal comprising parenterally injecting into the mammal a solution comprising the pharmaceutical composition according to Preferred embodiment 5 in a therapeutically effective amount sufficient for treating emesis or improving anesthesia recovery, the cyclodextrin being present in amounts that are sufficient for improving injection toleration at the injection site.

14. The method according to Preferred embodiment 13 wherein the cyclodextrin is about 2% to about 40% of the composition.

15. The method according to Preferred embodiment 14 wherein the cyclodextrin is about 4% to about 20% of the composition.

16. The method according to Preferred embodiment 15 wherein the cyclodextrin is about 5% to about 10% of the composition.

17. The method according to Preferred embodiment 16 wherein the cyclodextrin is sulfobutylether β-cyclodextrin or hydroxypropyl β-cyclodextrin.

18. The method according to Preferred embodiment 17 wherein the therapeutically effective amount of the NK-1 antagonist is 0.01 mg/kg to 100 mg/kg of a patient's body weight.

19. The method according to Preferred embodiment 18 wherein the therapeutically effective amount is 0.10 mg/kg to 10 mg/kg of a patient's body weight.

20. The method according to Preferred embodiment 19 wherein the pharmaceutically acceptable salt is citrate.

21. A method for improving injection site toleration during the treatment of emesis or improving anesthesia recovery in a mammal comprising parenterally injecting into the mammal an aqueous pharmaceutical solution of the pharmaceutical composition according to Preferred embodiment 5.

22. The method according to Preferred embodiment 21 wherein the cyclodextrin is about 2% to about 40% of the composition.

23. The method according to Preferred embodiment 22 wherein the cyclodextrin is about 4% to about 20% of the composition.

24. The method according to Preferred embodiment 23 wherein the cyclodextrin is about 5% to about 10% of the composition.

25. The method according to Preferred embodiment 24 wherein the cyclodextrin is sulfobutylether β-cyclodextrin or hydroxypropyl β-cyclodextrin.

26. The method according to Preferred embodiment 25 wherein the therapeutically effective amount of the NK-1 antagonist is 0.01 mg/kg to 100 mg/kg of a patient's body weight.

27. The method according to Preferred embodiment 26 wherein the therapeutically effective amount is 0.10 mg/kg to 10 mg/kg of a patient's body weight.

28. The method according to preferred embodiment 27 wherein the pharmaceutically acceptable salt is citrate.

1.-10. (canceled)

11. A pharmaceutical composition with an improved injection site toleration comprising a therapeutically effective amount of a neurokinin receptor (NK-1) antagonist with a pharmaceutically acceptable cyclodextrin.

12. A pharmaceutical composition according to claim 11 wherein the antagonist is selected from the group consisting of piperazine compounds, spiro-substituted azacycles, dialkyline piperadino compounds, trypthophan urea, polycyclic amine compounds, substituted arylaliphatic compounds, aromatic amine compounds, quaternary ammonium salts or aromatic amine compounds, aryl-substituted heterocycles, polycyclicamine compounds, substituted aryl piperazines, carboxamide derivatives, and bis-piperadinyl non-peptidal compounds, or salts thereof.

13. The pharmaceutical composition of claim 12 wherein the NK-1 antagonist is a compound comprising Formula I, 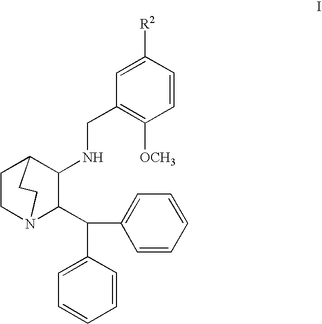
or pharmaceutically acceptable salt or prodrug thereof, wherein R² is selected from the group consisting of methyl, ethyl, isopropyl, sec-butyl and tert-butyl.

14. A pharmaceutical composition according to claim 13 wherein the compound comprising Formula I is a compound comprising Formula Ia, 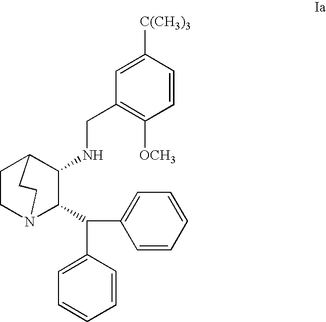
or a pharmaceutically acceptable salt or prodrug thereof.

15. A pharmaceutical composition according to claim 13 wherein the therapeutically effective amount of the NK-1 antagonist is 0.01 mg/kg to 100 mg/kg of a patient's body weight.

16. A pharmaceutical composition according to claim 14 wherein the therapeutically effective amount of the NK-1 antagonist is 0.01 mg/kg to 100 mg/kg of a patient's body weight.

17. A pharmaceutical composition according claim 15 wherein the therapeutically effective amount of the NK-1 antagonist is 0.10 mg/kg to 10 mg/kg of a patient's body weight.

18. A pharmaceutical composition according claim 16 wherein the therapeutically effective amount of the NK-1 antagonist is 0.10 mg/kg to 10 mg/kg of a patient's body weight.

19. The pharmaceutical composition according to claim 12 wherein said cyclodextrin is selected from β-cyclodextrin, sulfobutylether cyclodextrin, hydroxypropyl cyclodextrin, hydroxyethyl cyclodextrin, glucosyl cyclodextrin, maltosyl cyclodextrin, hydroxypropyl-β-cyclodextrin, sulfobutylether-β-cyclodextrin, hydroxyethyl-β-cyclodextrin, hydroxypropyl-γ-cyclodextrin, hydroxyethyl-β-cyclodextrin, dihydroxypropyl-β-cyclodextrin, glucosyl-β-cyclodextrin, diglycosyl-β-cyclodextrin, maltosyl-β-cyclodextrin, maltosyl-γ-cyclodextrin, maltotrialsyl-β-cyclodextrin, maltotrialsyl-γ-cyclodextrin, dimaltosyl-β-cyclodextrin, cyclodextrin derivatives, various mixtures of cyclodextrin derivatives thereof, mixtures such as maltosyl-β-cyclodextrin/dimaltosyl-β-cyclodextrin, and any other similar cyclodextrin known to those of skill in the art.

20. The pharmaceutical composition according to claim 19 wherein the cyclodextrin is selected from β-cyclodextrin, hydroxypropyl β-cyclodextrin, sulfobutylether β-cyclodextrin or substituted cyclodextrins.

21. The pharmaceutical composition according to claim 20 wherein the cyclodextrin is about 2% to about 40% of the composition.

22. The pharmaceutical composition according to claim 21 wherein the cyclodextrin is about 4% to about 20% of the composition.

23. The pharmaceutical composition according to claim 22 wherein the cyclodextrin is about 5% to about 10% of the composition.

24. The pharmaceutical composition according to claim 20 for use as a medicament.

25. The pharmaceutical composition of (2S,3S)-2-benzhydryl-N-(5-tert-butyl-2-methoxybenzyl)quinuclidin-3-amine and a pharmaceutically acceptable cyclodextrin where said cyclodextrin is selected from the group consisting of β-cyclodextrin, hydroxypropyl β-cyclodextrin, sulfobutylether β-cyclodextrin or substituted cyclodextrins.

26. The use of a composition according to claim 11 in the manufacture of a medicament for the treatment of a disease for which a NK-1 antagonist is indicated.

27. A method for the treatment of a disease for which a NK-1 antagonist is indicated in mammals comprising administering to said mammal a therapeutically effective amount of a pharmaceutical composition of claim 11.
